(19) United States
(12) Patent Application Publication
(10) Pub. No.: US 2021/0147890 A1
(43) Pub. Date: May 20, 2021

(54) COMPOSITION AND METHOD FOR DETERMINING THE AMOUNT OF GLUCOSE DERIVABLE FROM CELLULOSIC COMPONENTS OF FEEDSTOCK

(71) Applicant: DANISCO US INC., Palo Alto, CA (US)

(72) Inventor: Bradley R. Kelemen, Palo Alto, CA (US)

(21) Appl. No.: 16/623,916

(22) PCT Filed: Jul. 2, 2018
(86) PCT No.: PCT/US18/40562

§ 371 Date: Dec. 18, 2019

Related U.S. Application Data
(60) Provisional application No. 62/527,317, filed on Jun. 30, 2017.

(51) Int. Cl. C12P 19/02; C12N 9/42; C12P 19/14; C12P 7/10

(52) U.S. Cl. CPC C12P 19/02; C12N 9/2445; C12Y 302/01021; C12P 19/14; C12P 7/10; C12N 9/2437

(57) ABSTRACT

Described are compositions and methods relating to the measurement of glucose derivable from the cellulosic components of a feedstock. The compositions and methods are particularly useful for ensuring that biofuel producers comply with the Renewable Fuel Standard while maximizing the value of the biofuel they produce.

### TECHNICAL FIELD

The present compositions and methods relate to the precise and reliable measurement of the amount of glucose derived from cellulosic components in a feedstock. The compositions and methods are particularly useful for ensuring that biofuel producers comply with the Renewable Fuel Standard while maximizing the value of the biofuel they produce.

### BACKGROUND

The Renewable Fuel Standard (RFS) is a federal program that requires a minimum volume of renewable fuels to be blended into transportation fuel sold in the United States. The RFS originated with the Energy Policy Act of 2005 and was extended and expanded in the Energy Independence and Security Act of 2007. In 2010 the Environmental Protection Agency (EPA) established a process for companies to petition for new fuel pathways to qualify for the (RFS) program. A fuel pathway is a specific combination of (1) a feedstock, (2) a production process and (3) a fuel type, wherein each combination of three components represents a separate fuel pathway. Qualifying fuel pathways are assigned one or more D-codes corresponding to the type of Renewable Identification Number (RIN) they are eligible to generate. Conventional renewable fuel (e.g., from corn) is D6, advanced biofuel is D5, biodiesel is D4 and cellulosic biofuel is D3 or D7. Cellulosic Biofuel (D-Code 3 and 7) must be produced from cellulose, hemicellulose or lignin.

RINs are tradable regulatory credits that represent a quantity of qualifying renewable fuel. RINs are assigned after a producer reports the production of a gallon of fuel to the EPA. Blenders demonstrate compliance with the RFS by turning RINs over to the EPA once the gallon of fuel is blended into transportation fuel. Because the RFS requires increasing amounts of advanced biofuels (including cellulosic biofuels) at time progresses, RINs have different values depending on the fuel pathway from which they are generated. For example, a D3 RIN is worth more than a D6 RIN.

As corn ethanol producers attempt to utilize corn fiber, as well as corn starch, to produce ethanol, there is a financial incentive to characterize as much ethanol as possible as D3 biofuel. However, the EPA requires accuracy in accounting and producers that have non accurately characterized their biofuel can be subject to penalties. Accordingly, the need exists for an accurate method for determining the source of ethanol when mixed feedstocks of starch and cellulosic components are used to produce biofuels.

### SUMMARY

Described are compositions and methods relating to the measurement of the amount of glucose derived from the cellulosic components of a feedstock. Aspects and embodiments of the compositions and methods are described in the following, independently-numbered paragraphs.

1. In a first aspect, a method for measuring the amount of cellulose-derivable glucose in a feedstock comprising a mixture of starch and cellulosic components is provided, comprising: (i) contacting at least a portion of the feedstock with a cellulase composition deficient for β-D-glucoside glucohydrolase activity to obtain a partially-hydrolyzed slurry; (ii) contacting a portion of the partially-hydrolyzed slurry with an enzyme having β-D-glucoside glucohydrolase activity to produce glucose from cellulosic components present in the feedstock; (iii) measuring the amount of glucose present in the portion of the partially-hydrolyzed slurry contacted with the enzyme having β-D-glucoside glucohydrolase activity and the amount of glucose present in an otherwise identical portion of the partially-hydrolyzed slurry not contacted with an enzyme having β-D-glucoside glucohydrolase activity; and (iv) determining the amount of glucose derivable from cellulosic components in the feedstock based on the difference between the amount of glucose present in the portion of partially-hydrolyzed slurry contacted with the enzyme having β-D-glucoside glucohydrolase activity and present in the partially-hydrolyzed slurry not contacted with an enzyme having β-D-glucoside glucohydrolase activity.

2. In some embodiments of the method of paragraph 1, the amount of glucose present in the partially-hydrolyzed slurry contacted with the enzyme having β-D-glucoside glucohydrolase activity compared to the partially-hydrolyzed slurry not contacted with an enzyme having β-D-glucoside glucohydrolase activity is used to calculate the amount of ethanol derivable from cellulose components of the feedstock following fermentation with a fermenting organism.

3. In some embodiments of the method of paragraph 1, the feedstock is additionally contacted with a starch-hydrolyzing enzyme.

4. In some embodiments of the method of paragraph 3, the starch-hydrolyzing enzyme is an alpha-amylase and/or a glucoamylase.

5. In some embodiments of the method of paragraph 3 or 4, the difference in the amount of glucose present in the partially-hydrolyzed slurry contacted with the enzyme having β-D-glucoside glucohydrolase activity and the amount of glucose present in the partially-hydrolyzed slurry not contacted with an enzyme having β-D-glucoside glucohydrolase activity is used to calculate the fraction of ethanol that is derivable from cellulosic components of the feedstock following fermentation with a fermenting organism.

6. In some embodiments of the method of any of the preceding paragraphs, steps (ii)-(iv) are repeated on samples from (i) over a period of time.

7. In some embodiments of the method of any of the preceding paragraphs, the cellulase composition comprises cellobiohydrolase and endoglucanase.

8. In some embodiments of the method of any of the preceding paragraphs, the cellulase composition is deficient for β-glucosidase.

9. In some embodiments of the method of any of the preceding paragraphs, the enzyme having β-D-glucoside glucohydrolase activity is a β-glucosidase.

10. In some embodiments of the method of any of the preceding paragraphs, the partially-hydrolyzed slurry is manipulated to remove insoluble material.

11. In another aspect, a method for producing glucose from a feedstock comprising a mixture of starch and cellulosic components is provided, wherein the amount of glucose derived from the cellulosic components can be distinguished from the total amount of glucose derived from both the starch and cellulosic components of the feedstock, the method comprising: (i) contacting the feedstock with starch-hydrolyzing enzymes and a cellulase composition deficient for enzyme having β-D-glucoside glucohydrolase activity to obtain a partially-hydrolyzed slurry; (ii) determining the amount of glucose derivable from cellulosic components in the feedstock based on the difference in the amount of glucose present in a portion of the partially-hydrolyzed slurry contacted with an enzyme having β-D-glucoside glucohydrolase activity compared to the amount of glucose present in an otherwise identical soluble portion of the partially-hydrolyzed slurry not contacted with an enzyme having β-D-glucoside glucohydrolase activity; and (iii) adding an enzyme having β-D-glucoside glucohydrolase activity to the partially-hydrolyzed slurry; wherein the amount of glucose derived from the cellulosic components in the feedstock as a fraction of the total amount of glucose produced can be calculated based on the determination made in (ii).

12. In some embodiments of the method of paragraph 11, the amount of glucose present in the partially-hydrolyzed slurry contacted with the enzyme having β-D-glucoside glucohydrolase activity compared to the partially-hydrolyzed slurry not contacted with an enzyme having β-D-glucoside glucohydrolase activity is used to calculate the amount of ethanol derivable from cellulose components of the feedstock following fermentation with a fermenting organism.

13. In another aspect, a method for producing ethanol from a feedstock comprising a mixture of starch and cellulosic components is provided, wherein the amount of ethanol derived from the cellulosic components can be distinguished from the total amount of ethanol derived from both the starch and cellulosic components of the feedstock, the method comprising: (i) contacting the feedstock with starch-hydrolyzing enzymes and a cellulase composition deficient for enzyme having β-D-glucoside glucohydrolase activity to obtain a partially-hydrolyzed slurry; (ii) determining the amount of glucose derivable from cellulosic components in the feedstock based on the difference in the amount of glucose present in a portion of the partially-hydrolyzed slurry contacted with an enzyme having β-D-glucoside glucohydrolase activity compared to the amount of glucose present in an otherwise identical soluble portion of the partially-hydrolyzed slurry not contacted with an enzyme having β-D-glucoside glucohydrolase activity; (iii) adding an enzyme having β-D-glucoside glucohydrolase activity to the partially-hydrolyzed slurry; and (iv) contacting the partially-hydrolyzed slurry with a fermenting organism to produce ethanol, wherein the amount of ethanol derived from the cellulosic components in the feedstock as a fraction of the total amount of ethanol produced can be calculated based on the determination made in (ii).

14. In some embodiments of the method of paragraphs 11-13, the feedstock is additionally contacted with a starch-hydrolyzing enzyme.

15. In some embodiments of the method of paragraph 14, the starch-hydrolyzing enzyme is an alpha-amylase and/or a glucoamylase.

16. In some embodiments of the method of paragraphs 11-15, step (ii) is repeated on samples from (i) over a period of time.

17. In some embodiments of the method of paragraphs 11-16, the cellulase composition comprises cellobiohydrolase and endoglucanase.

18. In some embodiments of the method of paragraphs 11-17, the cellulase composition is deficient for β-glucosidase.

19. In some embodiments of the method of paragraphs 11-18, the enzyme having β-D-glucoside glucohydrolase activity is a β-glucosidase.

20. In another aspect, a two-component enzyme system for degrading cellulosic components in a biofuels feedstock is provided, comprising a first component composition comprising a mixture of cellulases deficient for β-D-glucoside glucohydrolase activity, wherein upon contact with soluble cellulase components in the feedstock the composition produces cellobiose and cello-oligosaccharides with substantially no monomer glucose, and a second component composition comprising an enzyme having β-D-glucoside glucohydrolase activity for producing glucose from cellobiose and cello-oligosaccharides.

21. In some embodiments of the system of paragraph 20, the first and/or second component composition comprises cellobiohydrolase and endoglucanase.

22. In some embodiments of the system of paragraph 20 or 21, the first component composition is deficient for β-glucosidase.

23. In some embodiments of the system of any of paragraphs 20-22, the first and/or second component composition further comprises a starch-hydrolyzing enzyme.

These and other aspects and embodiments of present modified cells and methods will be apparent from the description.

### BRIEF DESCRIPTION OF THE DRAWINGS

FIG. 1 is an HPLC chromatogram showing refractive index detection of separated sugars present in the supernatant of a sample collected at the end of fermentation. The dotted line indicates the initial sugar profile. The solid line shows the sugars present after half an hour of incubation with β-glucosidase. The peak eluting at the retention time of glucose (indicated) increases in the sample incubated for 30 minutes.

FIG. 2 is an HPLC chromatogram showing refractive index detection of separated sugars present in the supernatant of a sample collected at the end of fermentation. The dotted line shows the sugars present after half an hour incubation with β-glucosidase. The solid line shows the sugars present after an hour incubation with β-glucosidase. The peak eluting at the retention time of glucose (indicated) increases in the sample incubated for 60 minutes (30 minutes longer).

FIG. 3 is an HPLC chromatogram showing refractive index detection of separated sugars showing the retention time positions of cellobiose and glucose. The cellobiose peak (dotted line) resulted from incubating a cellobiose sample at 50° C. in buffer alone. The glucose peak (solid line) resulted from incubating the same starting cellobiose sample material with β-glucosidase for 30 minutes at 50° C.

FIG. 4 is an HPLC chromatogram showing refractive index detection of separated sugars showing the retention time position of maltose. The maltose peak (dotted line) resulted from incubating a maltose sample at 50° C. in buffer alone. The second maltose peak (solid line overlapping the dotted line) resulted from incubating the same starting maltose material with β-glucosidase for 30 minutes at 50° C.

### DETAILED DESCRIPTION

### I. Overview

The present compositions and methods are based on the ability to the precisely and reliably measure the amount of glucose derived or derivable from cellulosic components of a feedstock, including a mixed starch/cellulosic feedstock. The compositions and methods utilize a split, two-enzyme-component-system, wherein the first enzyme component generates cellobiose and cello-oligosaccharides from cellulosic components in the feedstock but does not generate monomeric glucose and the second enzyme component generates monomeric glucose from cellobiose and cello-oligosaccharides. The two-enzyme-component-system gives rise to an analytical method for determining the amount of glucose derivable from cellulosic components of a feedstock, as well as a full-scale method for producing glucose wherein the amount of glucose derivable from cellulosic components of a feedstock are known.

### II. Definitions

Prior to describing the present strains and methods in detail, the following terms are defined for clarity. Terms not defined should be accorded their ordinary meanings as used in the relevant art.

As used herein, “cellulose” refers to a polysaccharide consisting of a linear chain of β(1,4)-linked D-glucose units with the formula (C₆H₁₀O₅)ₙ, wherein “n” can be any number. Cellulose is a key structural component of plants.

As used herein, a “β-glucosidase” refers to an enzyme classified as EC 3.2.1.21 and having the activity of hydrolyzing terminal, non-reducing, β-D-glucosyl residues from cellulosic substrates to release monomeric β-D-glucose.

As used herein, “β-D-glucoside glucohydrolase” and “β-glucosidase activity” refers to the enzymatic activity of hydrolyzing terminal, non-reducing, β-D-glucosyl residues from cellulosic substrates to release monomeric β-D-glucose.

As used herein, a “cellobiohydrolase” refers to an enzyme classified as EC 3.2.1.91 and having the activity of hydrolyzing (1-4)-β-D-glucosidic linkages in cellulose and cellotetraose, releasing cellobiose and cello-oligosaccharides from the non-reducing ends of the chains.

As used herein, an “endocellulase” refers to an enzyme classified as EC 3.2.1.4 and having the activity of hydrolyzing internal bonds at amorphous sites in a cellulose substrate that create new chain ends.

As used herein, “starch” refers to a polysaccharide consisting of a linear chain of α(1,4) linked D-glucose units with the formula (C₆H₁₀O₅)ₙ, wherein “n” can be any number. Starch is abundant in grains, grasses, tubers and roots, and more specifically wheat, barley, corn, rye, rice, sorghum, brans, cassava, millet, potato, sweet potato, and tapioca.

As used herein, a “starch processing enzyme” is an enzyme that depolymerizes a starch substrate. Exemplary Starch processing enzymes are α-amylase, glucoamylase, β-amylase, pullulanase, and α-glucosidase.

As used herein, the phrase “degree of polymerization” (DP) refers to the number (n) of anhydroglucopyranose units in a given saccharide. Examples of DP1 are the monosaccharides glucose and fructose. Examples of DP2 are the disaccharides maltose and sucrose.

As used herein, a “feedstock” is a starting substrate for digestion to obtain glucose, which can optionally be used to produce valuable chemicals, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega 3 fatty acid, butanol, isoprene, 1,3-propanediol, pyruvate, 2,3-butanediol and other biomaterials, and alcohols, such as ethanol and butanol.

As used herein, a “partially-hydrolyzed slurry” is a product of treating a feedstock with digestive enzymes, typically under aqueous conditions and at elevated temperature, resulting in a semi-liquid mixture of soluble and insoluble materials.

As used herein, “contacting” an enzyme with a substrate refers to bringing the enzyme and substrate together in a common aqueous environment, typically accompanied by mixing to achieve uniform distribution. The term “contacted” is used interchangeably with “treated.”

As used herein, the phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

As used herein, an “ethanologenic microorganism” refers to a microorganism with the ability to convert a sugar or oligosaccharide to ethanol.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
g or gm grams
μg micrograms
mg milligrams
kg kilograms
μL and μl microliters
mL and ml milliliters
mm millimeters
μm micrometer
M molar
mM millimolar
μM micromolar
U units
min(s) minute/minutes
hr(s) hour/hours
HPLC high performance liquid chromatography

### III. Analytical Method

A first use of the present compositions and methods is an analytical method that can be used to determine how much glucose is derivable from the cellulosic components of a feedstock. The feedstock may be primarily cellulosic components (as in the case of a cellulosic ethanol facility) or a mixture of cellulosic and starch components, as in the case of a corn ethanol facility. A particular use of the compositions and methods is to determine the amount of ethanol derivable from the relatively small amount of cellulosic components (i.e., corn fiber) present in the feedstock used in corn ethanol facilities.

In practice, a feedstock is contacted with the first enzyme component, which is a cellulase composition that includes cellobiohydrolases, endoglucanases and other depolymerizing enzymes but is deficient for β-D-glucoside glucohydrolase activity. This deficiency may be a result of the absence or inactivation of β-glucosidase. The cellulase composition partially digests the cellulosic components in the feedstock to cellobiose and cello-oligosaccharides but does not produce monomeric glucose. This initial treatment of the feedstock can be on an analytical scale on a small portion of the feedstock that is to be converted on a larger scale. Alternatively, the initial treatment with the first enzyme component can represent the large treatment itself, where a small sample from the large scale treatment will subsequently be taken for use in the analytical method.

The first enzyme treatment may also include carbohydrate processing enzymes, including α-amylase and glucoamylase. Whether or not monomeric glucose is produced from starch components of the feedstock during or following the first enzyme treatment is not critical to the method. The first enzyme treatment may also include xylanase, protease, phytase, or essentially any other enzyme that does not have β-D-glucoside glucohydrolase activity. Enzymes such as protease and xylanase may be required to make the cellulosic components fully available.

After a suitable amount of time to allow the first enzyme composition to produce cellobiose and cello-oligosaccharides in what is now a slurry of partially-digested feedstock, a sample of the slurry is removed for analysis. In particular embodiments, the first enzyme treatment is performed for up to about 20, up to about, or even up to about 70 hours. The sample is split into two portions, one of which is treated with the second enzyme component and one of which is not. This part of the method is ideally performed on an analytic scale, even if the sample are taken from a large scale process. The remainder of the slurry may continue to process.

The second enzyme component, necessarily possesses robust β-D-glucoside glucohydrolase activity generate monomeric glucose from the cellobiose and cello-oligosaccharides produced by the first enzyme component. This second enzyme component is added to a portion of the slurry sample and a suitable “control” second component, e.g., having the same volume and containing the same formulation ingredients but not having β-D-glucoside glucohydrolase activity, is added to the other. The two portions of the slurry are now treated the same, being incubated for a time sufficient to allow glucose to be produced from cellobiose and cello-oligosaccharides. Ideally, this reaction should proceed to completion. In particular embodiments, the second enzyme treatment is performed for up to about 2, up to about, or even up to about 24 hours. Residual activity from the first enzyme component does not adversely affect the assay, nor does the addition of other enzymes, including additional cellobiohydrolase and endoglucanase.

The amount of glucose in the β-glucosidase-treated and untreated portions of the sample is measured using conventional analytical methods, such as HPLC, and the difference in the amount of glucose represents the amount of glucose from cellulosic components. This difference can be detected in the presence or absence of glucose produced from starch. The analysis can be repeated with further samples of the partially-digested feedstock over time.

Knowing the amount of glucose available from cellulosic components of the feedstock allows the determination of how much valuable end product can be attributed to the cellulosic component in the feedstock. Where amylases are included in the first enzyme treatment, the producer even knows the ratio of cellulosic-glucose to starch-glucose, and can simply apply this ratio to the total amount of end product produced.

While the method is described in its simplest form, numerous improvement can be made to increase accuracy and reproducibility. For example, β-glucosidase is known to work on soluble cellobiose and cello-oligosaccharides; therefore, it is preferred to remove insoluble materials from the portions of the sample to be treated (and untreated) with β-glucosidase. Insoluble materials are not compatible with soluble analytical techniques such as HPLC, therefore, insoluble material will likely have to be removed at some time prior to analysis. Insoluble material can be removed by, for example, filtration or centrifugation.

As noted above, samples from the first enzyme treatment may be taken over time until the amount of glucose generated with β-glucosidase no longer changes, indicating that substantially all the cellulose material has been accounted for. With experience, the analytical method will likely only be used when the first enzyme component has had sufficient time to substantially converted cellulosic materials in the feedstock to cellobiose and cello-oligosaccharides. In general, it is desirable to fully account for the amount of glucose derivable from cellulosic material in the feedstock.

Where carbohydrate processing enzymes are present in the first enzyme component, it may be desirable to ensure that that they have sufficient time to convert substantially all starch materials present in the feedstock to glucose, so that the analytical method is able to comprehensively determine the amounts and ratio of glucose derivable from cellulose and starch.

It may also be desirable to heat treat the sample from the first enzyme treatment, such that the enzymes from the first enzyme composition do not continue to change the partially-digested feedstock during treatment with the second enzyme composition. However, as noted above, this is not necessary as the continued activity of these enzymes is controlled for.

Where the first enzyme composition is applied on a large scale and samples are taken for use in the analytical method, β-glucosidase must eventually be added to partially digested slurry to liberate glucose from the remainder of the cellobiose and cello-oligosaccharides. Production facilities will want to do this as soon as possible. With experience using the method, this could be performed as soon as a sample (or series of samples) are taken for the analytical method (which assumes that the first enzyme treatment has adequately converted the cellulosic material to cellobiose and cello-oligosaccharides), or after multiple samples are taken and analyzed to be certain that the first enzyme treatment has converted essentially all the cellulosic components to cellobiose and cello-oligosaccharides. The latter situation is likely until the production facility knows how the feedstock will behave after being contacted with the first enzyme component. The former situation is more likely once the production facility gains experience with the application of the method. Samples can also be taken and analyzed at a later time.

In a particular embodiment, the analytical method can be used to accurately determine how much cellulose-derived ethanol is made from any given feedstock. This will allow a biofuel producer to maximize the value of biofuel produced from a given feedstock under the Renewable Fuel Standard (RFS).

### III. Method for Producing Glucose

A second use of the present compositions and methods is a full-scale method for producing glucose, wherein the amount of glucose derivable from the cellulosic components of a feedstock is known. Unlike the above-described analytical method, which represents a tool that can be used by biochemical producers to extract more value from their product, this embodiment represents an improvement in the actual process of producing glucose from a feedstock on a large scale.

In practice, a feedstock is contacted with the first enzyme component on a full-scale basis (i.e., the normal scale of a production facility). After a suitable amount of time to allow the first enzyme composition to produce cellobiose and cello-oligosaccharides in what is now a slurry of partially-digested feedstock, a sample of the slurry is removed for analysis. In particular embodiments, the first enzyme treatment is performed for up to about 20, up to about, or even up to about 70 hours. As before the sample is split into two portions, one of which is treated with the second enzyme component and one of which is not. This part of the method is still ideally performed on an analytic scale.

Where a mixed cellulosic component/starch feedstock is used, it is desirable to include starch processing enzymes, including α-amylases and glucoamylases to digest substantially all starch in the feedstock to glucose at the same time cellulosic components are being converted to cellobiose and cello-oligosaccharides. Additional enzymes such as protease and xylanase may also be used to make as much of the cellulose and starch available as possible.

Samples from the first enzyme treatment are likely to be taken at multiple times following contact with the first enzyme composition, until the amount of glucose generated with β-glucosidase does not change, indicating that substantially all the cellulose components have been accounted for.

The second enzyme component must be added to the bulk of the partially digested slurry as soon as possible to liberate glucose from the remainder of the cellobiose and cello-oligosaccharides. With experience using the method, this could be performed as soon as a sample (or series of samples) are taken for treatment with the second enzyme composition (which assumes that the first enzyme treatment has converted essentially all the cellulosic components to cellobiose and cello-oligosaccharides), or after multiple samples are taken and analyzed to be certain that the first enzyme treatment has converted essentially all the cellulosic components to cellobiose and cello-oligosaccharides. As with the analytical method, experience will dictate when samples are taken and, in this case, when β-glucosidase is added to the bulk of the slurry.

The large-scale method can be used to produce glucose and subsequent products which glucose can be attributed to either cellulosic components or starch in the feedstock. Where the glucose is fermented to ethanol, the method allows an ethanol producer to accurately and reliably determine the assignment of RINs to the batch of ethanol produced from any given feedstock, maximize the value of the ethanol under the Renewable Fuel Standard (RFS).

### III. Two Component Enzyme System

The present compositions and methods further give rise to a two enzyme component enzyme system for producing glucose and subsequent end products from a feedstock containing cellulosic components, including a mixed cellulose/starch feedstock.

The first enzyme component is a cellulase composition that includes cellobiohydrolase and endoglucanase but is deficient for β-D-glucoside glucohydrolase activity. This deficiency may be a result of the absence or inactivation of β-glucosidase. Preferably, the first enzyme component has less than 10%, less than 5%, and even less than 1% of the relative units of β-D-glucoside glucohydrolase activity compared to a whole cellulose broth from a wood degrading fungi, such as Trichoderma. β-glucosidase activity can be determined by any means know in the art, such as the assay described by Chen, H. et al. ((1992) Biochimica et Biophysica Acta, 121:54-60), wherein one para-nitrophenyl β-D-glucoside (pNPG) unit denotes 1 μmol of nitrophenol liberated from para-nitrophenyl-B-D-glucopyranoside in 10 minutes at 50° C. and pH 4.8. β-D-glucoside glucohydrolase activity can also be determined as described in US2010221784 and US2013337508, which are incorporated by reference. These documents also describe the typical levels of β-D-glucoside glucohydrolase activity present in whole cellulase broths.

The cellulase composition may further include a starch processing enzyme such as α-amylase, glucoamylase, β-amylase, pullulanase, and α-glucosidase as well as other enzymes, such as xylanase, protease, phytase, and other enzymes that do not have β-D-glucoside glucohydrolase activity.

The second enzyme component includes a β-glucosidase may further include any of the enzymes present in the first enzyme component. In some embodiment, the second enzyme component includes only a β-glucosidase.

### IV. Enzymes and Feedstocks

All enzymes and enzyme activities described herein are available in the form of widely available commercial products, including many that are currently used in carbohydrate and cellulose processing and/or biofuel or other biochemical production. The present compositions and methods are in no way reliant on enzymes defined by a source organism or by amino acid sequence identity. The present compositions and methods are based on enzyme specificity, not specific enzymes.

The compositions and methods are not limited to the use of a particular feedstock. The feedstock logically comprises cellulose components from which glucose may be derived but may be from any plant material and may include any amount, including a majority, of starch material.

These and other aspects and embodiments of the present strains and methods will be apparent to the skilled person in view of the present description. The following examples are intended to further illustrate, but not limit, the strains and methods.

### EXAMPLES

### Example 1: Sensitivity of Supernatant from End of Fermentation to β-Glucosidase Activity

A disaccharide (DP2) was observed with chromatographic retention time similar to that of either maltose or cellobiose in the supernatant of a sample taken from the end of fermentation (fermentation drop) of a grain ethanol process. Few chromatographic methods can distinguish cellobiose, derived from cellulose, from maltose, derived from starch. The following treatment with a purified β-glucosidase establishes that a portion of the chromatographic peak for that disaccharide originated from cellulose.

A sample (500 g) of whole slurry fermentation drop was collected from a sampling port in a full-scale ethanol production facility. The sample was frozen to preserve for future analysis. The sample was thawed and a smaller sample (1 g) was subjected to centrifugation to remove the insoluble solid material. 0.100 ml of the resulting supernatant was combined with 0.100 ml 50 mM sodium acetate buffer at pH 5 in each of two tubes. A small volume (0.001 ml) of purified β-glucosidase (4.5 mg/ml) was added to one of those tubes.

0.100 ml of a solution of cellobiose (20 mM) was combined with of 50 mM sodium acetate. A small volume (0.001 ml) of purified β-glucosidase (4.5 mg/ml) was added to one of those tubes. The reactions were placed at 50° C. for overnight incubation.

All the reactions were then diluted with 10 mM sulfuric acid and filtered through 0.2 μm filter for analysis by HPLC. Quantitation standards were similarly diluted with 10 mM sulfuric acid and filtered for analysis by HPLC.

Incubation of supernatant from fermentation drop with buffer at 50° C. overnight resulted in 3.1 mg/ml glucose as determined by HPLC. Incubation of supernatant from fermentation drop with β-glucosidase at 50° C. overnight resulted in 4.4 mg/ml glucose as determined by HPLC. The portion of cellooligosaccharides available in the fermentation drop sample is the difference between these values, i.e., 4.4-3.1 mg/ml=the equivalent of 1.3 mg/ml of glucose.

### Example 2: Shorter Incubation Times Indicate Catalytic Formation of Glucose

Supernatant of fermentation drop (0.100 ml) was prepared as described in Example 1. A solution of maltose (20 mM, 0.100 ml), and a solution of cellobiose (20 mM, 0.100 ml) were individually combined with 50 mM sodium acetate buffer at pH 5 (0.100 ml) in two tubes for each sample. A small volume (0.001 ml) of purified β-glucosidase (4.5 mg/ml) was added to one of each pair of tubes. The reactions were incubated at 50° C.

A portion (0.075 ml) of each sample was removed after half an hour and one hour and combined with (0.075 ml) of 10 m sulfuric acid. Sugar standards were prepared in a similar manner as the samples. The samples were analyzed by HPLC.

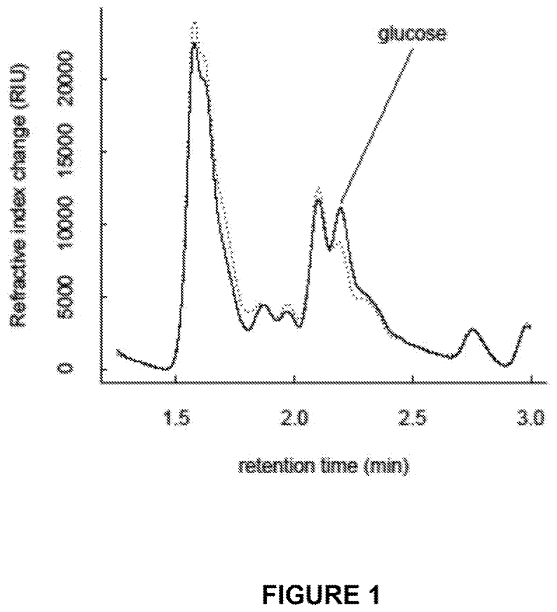

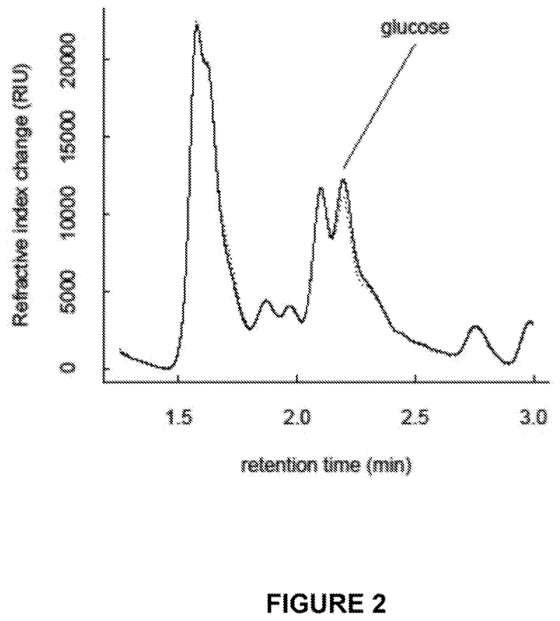

As shown in the chromatogram in FIG. 1, the amount of glucose increases with the addition of β-glucosidase from initial conditions to incubation for 30 minutes at 50° C. (FIG. 1). As shown in the chromatogram in FIG. 2, the amount of glucose further increases with the addition of β-glucosidase for an addition 30 minutes (60 minutes total).

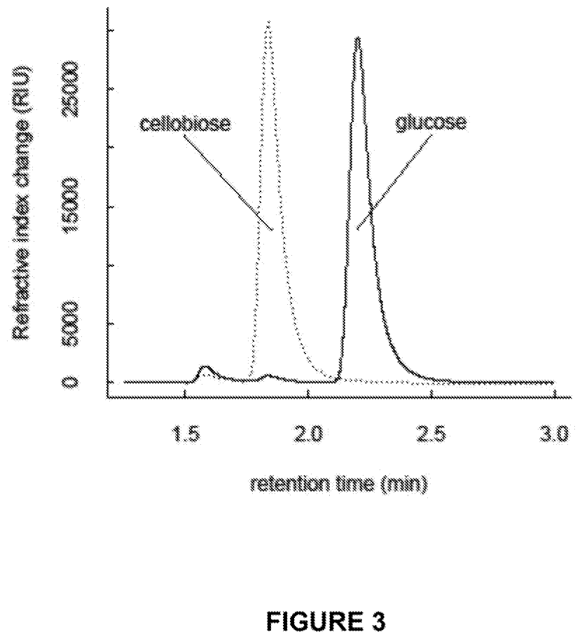

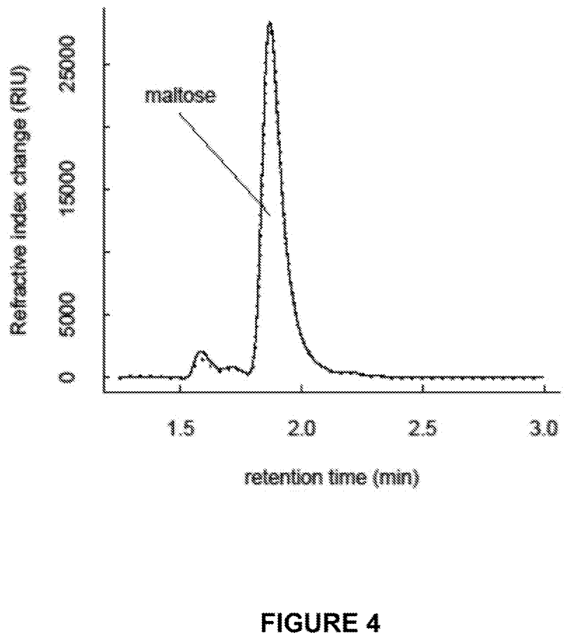

As shown in the chromatogram in FIGS. 3 and 4, all cellobiose is converted to glucose (FIG. 3) and no maltose is converted to glucose (FIG. 4) by the same preparation of β-glucosidase.

What is claimed is:

1. A method for measuring the amount of cellulose-derivable glucose in a feedstock comprising a mixture of starch and cellulosic components, comprising:
(i) contacting at least a portion of the feedstock with a cellulase composition deficient for β-D-glucoside glucohydrolase activity to obtain a partially-hydrolyzed slurry;
(ii) contacting a portion of the partially-hydrolyzed slurry with an enzyme having β-D-glucoside glucohydrolase activity to produce glucose from cellulosic components present in the feedstock;
(iii) measuring the amount of glucose present in the portion of the partially-hydrolyzed slurry contacted with the enzyme having β-D-glucoside glucohydrolase activity and the amount of glucose present in an otherwise identical portion of the partially-hydrolyzed slurry not contacted with an enzyme having β-D-glucoside glucohydrolase activity; and
(iv) determining the amount of glucose derivable from cellulosic components in the feedstock based on the difference between the amount of glucose present in the portion of partially-hydrolyzed slurry contacted with the enzyme having β-D-glucoside glucohydrolase activity and present in the partially-hydrolyzed slurry not contacted with an enzyme having β-D-glucoside glucohydrolase activity.

2. The method of claim 1, wherein the amount of glucose present in the partially-hydrolyzed slurry contacted with the enzyme having β-D-glucoside glucohydrolase activity compared to the partially-hydrolyzed slurry not contacted with an enzyme having β-D-glucoside glucohydrolase activity is used to calculate the amount of ethanol derivable from cellulose components of the feedstock following fermentation with a fermenting organism.

3. The method of claim 1, wherein the feedstock is additionally contacted with a starch-hydrolyzing enzyme.

4. The method of claim 3, wherein the starch-hydrolyzing enzyme is an alpha-amylase and/or a glucoamylase.

5. The method of claim 3 or 4, wherein difference in the amount of glucose present in the partially-hydrolyzed slurry contacted with the enzyme having β-D-glucoside glucohydrolase activity and the amount of glucose present in the partially-hydrolyzed slurry not contacted with an enzyme having β-D-glucoside glucohydrolase activity is used to calculate the fraction of ethanol that is derivable from cellulosic components of the feedstock following fermentation with a fermenting organism.

6. The method of any of the preceding claims, wherein steps (ii)-(iv) are repeated on samples from (i) over a period of time.

7. The method of any of the preceding claims, wherein the cellulase composition comprises cellobiohydrolase and endoglucanase.

8. The method of any of the preceding claims, wherein the cellulase composition is deficient for β-glucosidase.

9. The method of any of the preceding claims, wherein the enzyme having β-D-glucoside glucohydrolase activity is a β-glucosidase.

10. The method of any of the preceding claims, wherein the partially-hydrolyzed slurry is manipulated to remove insoluble material.

11. A method for producing glucose from a feedstock comprising a mixture of starch and cellulosic components, wherein the amount of glucose derived from the cellulosic components can be distinguished from the total amount of glucose derived from both the starch and cellulosic components of the feedstock, the method comprising:
(i) contacting the feedstock with starch-hydrolyzing enzymes and a cellulase composition deficient for enzyme having β-D-glucoside glucohydrolase activity to obtain a partially-hydrolyzed slurry;
(ii) determining the amount of glucose derivable from cellulosic components in the feedstock based on the difference in the amount of glucose present in a portion of the partially-hydrolyzed slurry contacted with an enzyme having β-D-glucoside glucohydrolase activity compared to the amount of glucose present in an otherwise identical soluble portion of the partially-hydrolyzed slurry not contacted with an enzyme having β-D-glucoside glucohydrolase activity; and
(iii) adding an enzyme having β-D-glucoside glucohydrolase activity to the partially-hydrolyzed slurry;
wherein the amount of glucose derived from the cellulosic components in the feedstock as a fraction of the total amount of glucose produced can be calculated based on the determination made in (ii).

12. The method of claim 11, wherein the amount of glucose present in the partially-hydrolyzed slurry contacted with the enzyme having β-D-glucoside glucohydrolase activity compared to the partially-hydrolyzed slurry not contacted with an enzyme having β-D-glucoside glucohydrolase activity is used to calculate the amount of ethanol derivable from cellulose components of the feedstock following fermentation with a fermenting organism.

13. A method for producing ethanol from a feedstock comprising a mixture of starch and cellulosic components, wherein the amount of ethanol derived from the cellulosic components can be distinguished from the total amount of ethanol derived from both the starch and cellulosic components of the feedstock, the method comprising:
(i) contacting the feedstock with starch-hydrolyzing enzymes and a cellulase composition deficient for enzyme having β-D-glucoside glucohydrolase activity to obtain a partially-hydrolyzed slurry;
(ii) determining the amount of glucose derivable from cellulosic components in the feedstock based on the difference in the amount of glucose present in a portion of the partially-hydrolyzed slurry contacted with an enzyme having β-D-glucoside glucohydrolase activity compared to the amount of glucose present in an otherwise identical soluble portion of the partially-hydrolyzed slurry not contacted with an enzyme having β-D-glucoside glucohydrolase activity;
(iii) adding an enzyme having β-D-glucoside glucohydrolase activity to the partially-hydrolyzed slurry; and
(iv) contacting the partially-hydrolyzed slurry with a fermenting organism to produce ethanol,
wherein the amount of ethanol derived from the cellulosic components in the feedstock as a fraction of the total amount of ethanol produced can be calculated based on the determination made in (ii).

14. The method of any of claims 11-13, wherein the feedstock is additionally contacted with a starch-hydrolyzing enzyme.

15. The method of claim 14, wherein the starch-hydrolyzing enzyme is an alpha-amylase and/or a glucoamylase.

16. The method of any of claims 11-15, wherein step (ii) is repeated on samples from (i) over a period of time.

17. The method of any of claims 11-16, wherein the cellulase composition comprises cellobiohydrolase and endoglucanase.

18. The method of any of claims 11-17, wherein the cellulase composition is deficient for β-glucosidase.

19. The method of any of claims 11-18, wherein the enzyme having β-D-glucoside glucohydrolase activity is a β-glucosidase.

20. A two-component enzyme system for degrading cellulosic components in a biofuels feedstock, comprising a first component composition comprising a mixture of cellulases deficient for β-D-glucoside glucohydrolase activity, wherein upon contact with soluble cellulase components in the feedstock the composition produces cellobiose and cello-oligosaccharides with substantially no monomer glucose, and a second component composition comprising an enzyme having β-D-glucoside glucohydrolase activity for producing glucose from cellobiose and cello-oligosaccharides.

21. The system of claim 20, wherein the first and/or second component composition comprises cellobiohydrolase and endoglucanase.

22. The system of claim 20 or 21, wherein the first component composition is deficient for β-glucosidase.

23. The system of any of claims 20-22, wherein the first and/or second component composition further comprises a starch-hydrolyzing enzyme.
